(19) United States
(12) Patent Application Publication
(10) Pub. No.: US 2002/0078961 A1
(43) Pub. Date: Jun. 27, 2002

(54) Laryngeal mask assemblies

(76) Inventor: Michael Norman Collins, Lyminge (GB)

Correspondence Address: LAW OFFICES OF LOUIS WOO, Suite 501, 1901 North Fort Myer Drive, Arlington, VA, 22209, US

(21) Appl. No.: 09/989,393

(22) Filed: Nov. 21, 2001

(30) Foreign Application Priority Data
Dec. 22, 2000 (GB) 0031661.2

(51) Int. Cl. A61M 16/00; A62B 9/06

(52) U.S. Cl. 128/207.15; 128/200.26; 128/207.14

(57) ABSTRACT

A laryngeal mask has a tube and mount integrally molded from polyurethane as a single piece. A sealing cuff is attached to the mount separately. An inflation line extends in a groove along the outside of the tube and opens at one end into the cuff so that it can be inflated and deflated.

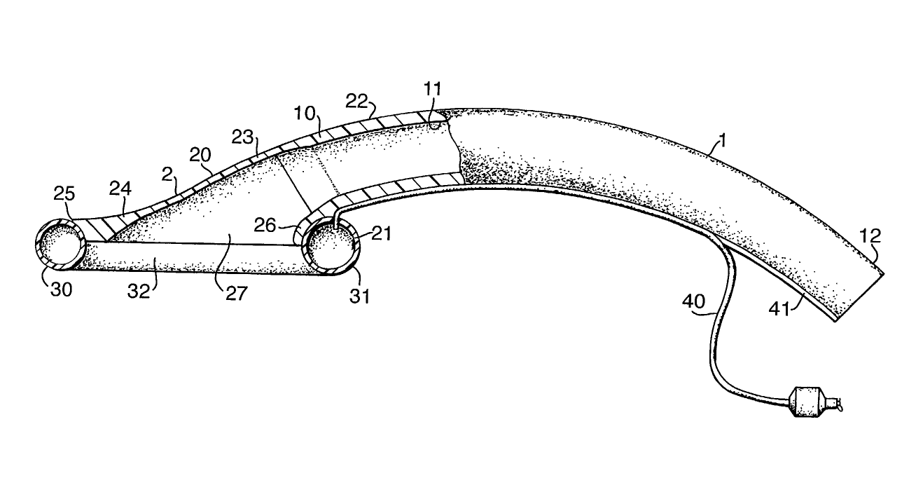

### BACKGROUND OF THE INVENTION

This invention relates to laryngeal mask assemblies and their manufacture

It is common practice to use an airway known as a laryngeal mask for administering anaesthetic and ventilation gases to a patient. These airways comprise a tube with an inflatable mask or cuff at one end, the tube being inserted in the patient's mouth so that one end is located in the hypopharynx and so that the mask forms a seal in this region with the surrounding tissue. Laryngeal masks are described in, for example, U.S. Pat. Nos. 5,355,879, 5305743, 5297547, 5282464, GB 2267034, U.S. Pat. Nos. 5,249,571, 5,241,956, 5,303,697, GB 2249959, GB 2111394, EP 448878, U.S. Pat. No. 4,995,388, GB 2205499, GB 2128561, GB 2298797, GB 2334215, GB2337020, PCT/GB00/03044, PCT/GB00/03045, GB 0002805 and GB 0020274. Laryngeal masks usually comprise a curved, extruded tube, a separate mount member joined at the patient end of the tube and an inflatable cuff attached to the mount member.

Laryngeal masks have several advantages over endotracheal tubes, which are longer and seal with the trachea below the vocal folds. The multiple components and assembly operations needed to make the masks, however, add to their cost.

It is an object of the present invention to provide an alternative laryngeal mask assembly.

According to one aspect of the present invention there is provided a laryngeal mask assembly comprising a tube, a mount at the patient end of the tube, and an annular sealing cuff extending around the patient end of the mount, the tube and mount being molded together as an integral, single-piece component.

The sealing cuff may be attached with the mount by an adhesive. The sealing cuff is preferably inflatable and deflatable by means of an inflation line, the inflation line extending in a groove along the outside of the tube. The tube and mount may be molded of polyurethane.

According to another aspect of the present invention there is provided a method of manufacture of a laryngeal mask assembly comprising the steps of molding a tube and a mount integrally with the tube and subsequently attaching a sealing cuff with the mount.

According to a further aspect of the present invention there is provided a laryngeal mask assembly made by the method of the above other aspect of the invention.

A laryngeal mask assembly according to the present invention will now be described, by way of example, with reference to the accompanying drawing.

### BRIEF DESCRIPTION OF THE DRAWING

The drawing is a side elevation view of the assembly.

### DETAILED DESCRIPTION OF THE PREFERRED EMBODIMENT

The laryngeal mask assembly comprises a tube 1 and a mask formation 2 at the patient end 10 of the tube.

The tube 1 is of a bendable plastics material, such as PVC and is curved along its length. A bore 11 extends along the tube from its patient end 10 to its rear, machine end 12.

The mask 2 comprises a mount 20 and an inflatable sealing cuff 21. The mount 20 is of a relatively stiff plastics material and is of generally shoe shape. The mount 20 and tube 1 are molded together, such as by injection molding, to form an integral, single piece 22. The mount 20 tapers outwardly from its machine end 23 to its patient end 24, which is inclined to the axis of the machine end at an angle of about 25° so that the patient end of the mount has an oval shape with its forward end 25 being more pointed than its rear end 26. The patient end 24 of the mount 20 is inclined to face towards the inner side of the curve of the tube 1. Internally, the mount 20 has a cavity 27 that increases in cross-sectional area along its length, from the machine end.

The cuff 21 is tubular and of a thin flexible plastics material. The cuff 21 is formed into an annulus of the same shape as the patient end 24 of the mount 20 so that it is oval with its forwardly-directed end 30 being more pointed than its rearwardly-directed end 31. The cuff 21 encloses a central region 32 of the same shape as the patient end 24 of the mount 20. The cuff 21 is attached around the patient end 24 of the mount 20 such as by means of an adhesive. The cuff 21 is inflated and deflated by means of an inflation line 40 which is provided by a separate small-bore tube communicating with the interior of the cuff and extending rearwardly along a groove 41 in the outside of the tube. When inflated in position in a patient, the cuff 21 expands to contact patient tissue in the region of the hypopharnyx.

Because the mount and tube are formed in one operation, there are fewer separate components and fewer steps needed to manufacture the assembly. By avoiding the need to bond the mount to the tube, there is no risk of a faulty bond and there is no need to inspect a bond. It is possible to achieve a smooth external profile in the region between the tube and mount, without a step or bump, because there is no connection in this region. Molding the tube and mount together enables the wall thickness or shape to be varied, if desired, at different points along the length of the tube. It is also possible, by molding, to use different materials, such as polyurethane, which present problems with extrusion.

What I claim is:

1. A laryngeal mask assembly comprising: a tube; a mount at a patient end of said tube; and an annular sealing cuff extending around a patient end of said mount, wherein said tube and mount are molded together as an integral, single-piece component.

2. A laryngeal mask assembly according to claim 1, wherein said sealing cuff is attached with said mount by an adhesive.

3. A laryngeal mask assembly according to claim 1 including an inflation line opening at one end into said sealing cuff, wherein said inflation line extends in a groove along an outside of said tube, and wherein said sealing cuff is inflatable and deflatable via said inflation line.

4. A laryngeal mask assembly according to claim 1, wherein said tube and mount are molded of polyurethane.

5. A laryngeal mask assembly comprising: a tube; a mount at a patient end of said tube; an inflation line extending in a groove along said tube; an annular sealing cuff extending around a patient end of said mount in communication with said inflation line, wherein said tube and mount are molded together as an integral, single-piece component, and wherein said sealing cuff is attached with said mount as a separate component and is adapted to seal with tissue in the region of the hypopharynx.

6. A method of manufacture of a laryngeal mask assembly comprising the steps of: molding a tube and a mount integrally with said tube; and subsequently attaching a sealing cuff with said mount.

7. A method of manufacture of a laryngeal mask assembly comprising the steps of: molding a tube and a mount integrally with said tube, said mount being of generally shoe-shape and having a patient end extending at an angle to an axis of the tube; and subsequently attaching a sealing cuff with said mount, said mount being shaped such that said cuff can seal with tissue in the region of the hypopharynx.
